(19) United States
(12) Patent Application Publication
(10) Pub. No.: US 2012/0253236 A1
(43) Pub. Date: Oct. 4, 2012

(54) METHODS AND APPARATUSES FOR DELIVERING EXTERNAL THERAPEUTIC STIMULATION TO ANIMALS AND HUMANS

(76) Inventors: Buddy L. Snow, Wayzata, MN (US); Steven D. Moe, Savage, MN (US)

(21) Appl. No.: 13/423,426

(22) Filed: Mar. 19, 2012

Related U.S. Application Data
(60) Provisional application No. 61/471,379, filed on Apr. 4, 2011.

(51) Int. Cl. A61H 1/00; A61N 5/06; A61N 7/00

(52) U.S. Cl. 601/2; 607/88; 601/46; 601/47

(57) ABSTRACT

This invention described herein includes methods and apparatuses for externally delivering therapeutic stimulation to animals and humans to help improve their health, condition and performance. The invention includes various wearable, attachable, and embeddable devices that deliver multiple forms of therapeutic stimulation or vibration such as mechanical, tonal, audio, light, and electrical pulses.

### BACKGROUND OF THE INVENTION

The invention is in the field of external therapeutic stimulation or delivery of energy to animals or humans.

Today it is well known to provide humans and animals with vibrational therapy through massage chairs, hand massagers, whole body vibration plates, handheld lasers or similar apparatuses that are applied to different parts of the body, sometimes with or without heat. These devices have served to relax the individual by stimulating the nervous system and promoting blood flow and increased oxygen to a particular part of the body to help with conditions and areas such as anxiety, post-operative healing, athletic and general performance, pain, aging, injuries, obesity, and general health. It would be desirable to provide similar therapy options to pets, humans and animals that are convenient and easy to use on a frequent basis, as the brain, nervous system, and body are in a constant state of change and all benefit from new stimulation.

### SUMMARY OF THE INVENTION

The teachings of the invention relate to simple and constant focused delivery of external stimulation or energy via vibration, tones, audio, electrical, pulse, light or other similar sources, through embedded leads, conductors or other similar methods and devices to certain portions of a body. The various example embodiments described herein incorporate the various stimulation therapies in convenient, easy to use form factors that include wearable, embeddable or attachable forms. Additionally, the invention can incorporate wireless, remote and direct communication technology to interact with and direct/control the therapeutic device that has already been placed on an animal. In various embodiments, the teachings of the invention are incorporated into an external clip, blanket, collar, harness, headband, wrap, shirt, bedding, crate or other similar delivery method with internal leads, extenders, conductors, or similar methods, to deliver a consistent and constant source of energy to animals. In a related embodiment, the invention also includes methods of delivering consistent forms of energy such as vibration, tones, audio, electrical, pulse, light or other similar sources. In yet another embodiment, the invention includes ways of remotely, directly, wirelessly or other similar ways of interacting and operating a device placed on an animal.

In one example embodiment, an apparatus and method of delivering focused or directional low magnitude, high frequency therapeutic energy or stimulation invariable, attachable and/or embeddable forms, that brings about high vibration in the subject's tissue or bone, including sources such as vibration, tones, audio, electrical, pulse, light or other similar forms, directly in forms including clips, attachments, collars, straps, blankets, wraps, shirts, beds, crates, or other similar methods, externally to animals, mammals or humans are disclosed herein.

In another example embodiment, an apparatus and method of remotely, wirelessly, directly, or through other similar handheld devices or technology, interacting and operating a device that brings about high vibration in the subject's tissue or bone located or placed on an animal or other mammals are disclosed herein.

In yet another example embodiment, an apparatus and method of delivering various sources of therapeutic or stimulation energy that brings about high vibration in the subject's tissue or bone such as vibration, tones, audio, electrical, pulse, light or other similar forms, constantly and consistently throughout materials placed on or around animals or mammals are disclosed herein.

### BRIEF DESCRIPTION OF DRAWINGS

FIG. 1 is a front view of a collar, belt, necklace, halter, harness, bracelet, strap or similar wearable method, with a control panel consisting of a controlling mechanism, according to the teachings of the invention.

FIG. 2 is a top view of the collar in FIG. 1.

FIG. 3 is a front view of another device for delivering external energy via a clip generator according to the teachings of the invention.

FIG. 4 is a side view of the clip generator of FIG. 3.

FIG. 5 is a front view of another strap or collar with more prominent speakers, control panel, and with internal leads running throughout, according to the teachings of the invention.

FIG. 6 is a blanket, wrap, shirt or other similar method, with control panel inserted into it, according to the teachings of the invention.

FIG. 7 is a side view of the wrap of FIG. 6.

FIG. 8 is a system of communicating remotely with the collar of FIG. 1, from any location through a handheld or similar communication device, according to the teachings of the invention.

FIG. 9 is a device for attaching or embedding the control unit into a bed, mattress or similar item where an animal spends time sitting or lying down, according to the teachings of the invention.

FIG. 10 is a method of embedding the control unit into an animal crate or similar enclosure, according to the teachings of the invention.

FIG. 11 shows methods of attaching a clip, and control unit embedded into a belt, wrap, or headband to a human, according to the teachings of the invention.

FIGS. 12A-12C shows in further detail different ways a clip can be attached to belts, collars, bands, caps or similar areas according to the teachings of the invention.

FIG. 13 is a detailed view of the control panel, and the components that can be included therein, according to the teachings of the invention herein.

### DETAILED DESCRIPTION OF THE INVENTION

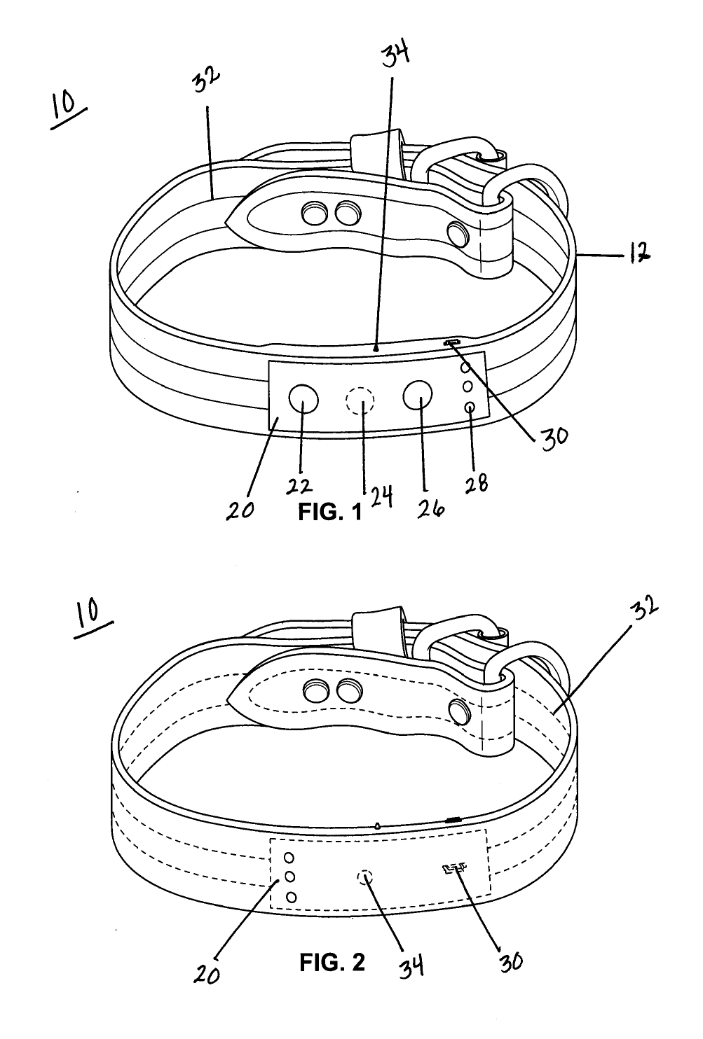

Referring now to the invention in more detail, FIGS. 1 and 2 illustrate an external, wearable, stimulation device 10 configured to deliver consistent and focused energy to animals or mammals (or humans). These types of embedded sources of energy can include, but are not limited to, vibration, tones, audio, electrical, pulse, light or the like. Various embodiments include a collar 12, belt, necklace, halter, harness, bracelet, strap or the like that is attached to the recipient around its neck or other body part. FIG. 1 includes a wireless port 34 to interact remotely, speaker(s) 24, or other mechanical form of vibrational energy, inserted into it to deliver a consistent source of energy. In this embodiment, internal leads, extenders, conduction material, conductors 32, or the like are deployed to deliver energy throughout device 10.

Inserted into the collar or strap, in various embodiments described herein, is a fully-enclosed, waterproof, safe, control panel 20 consisting of components such as a:
Power button 22—to turn the control panel on or off
Speaker 24—to deliver tones or audio
Laser housing—to deliver intermittent laser therapy (optional)
Vibrator and motor—to deliver manual vibration
Mode selector button 26—to select the mode of energy source (vibration, tones, audio, electrical, pulse, light or similar)
Indicator lights 28—showing which stimulation method is selected or active
Plug-in port 30—for external power (or battery) or audio loading
Battery—which may also be removable and rechargeable to power the tone/vibration/audio generator device (not shown)
Wireless port 34—to remotely connect to device

In the various embodiments, collar or strap 10 may have leads or other transmittal components 32 embedded into it to deliver energy from control panel 20 through entire device 10.

In more detail, still referring to FIGS. 1 and 2, when the user turns on device 10, consistent energy in the form of vibration, tones, pulse, laser, or other therapeutic energy is delivered to the animal (or mammal) for a set period of time. The user can manually turn off device 10 or it will turn off automatically at a set time period.

In the various embodiments, the vibration, tones, pulse, laser, or audio functions will be available in multiple levels that can be controlled via mode button 26.

The construction details of the invention in FIGS. 1 and 2 include collar or strap 12, being made from nylon, leather or other material, and control panel 20 being fully enclosed in a unit made of plastic, metal, leather, fabric or the like.

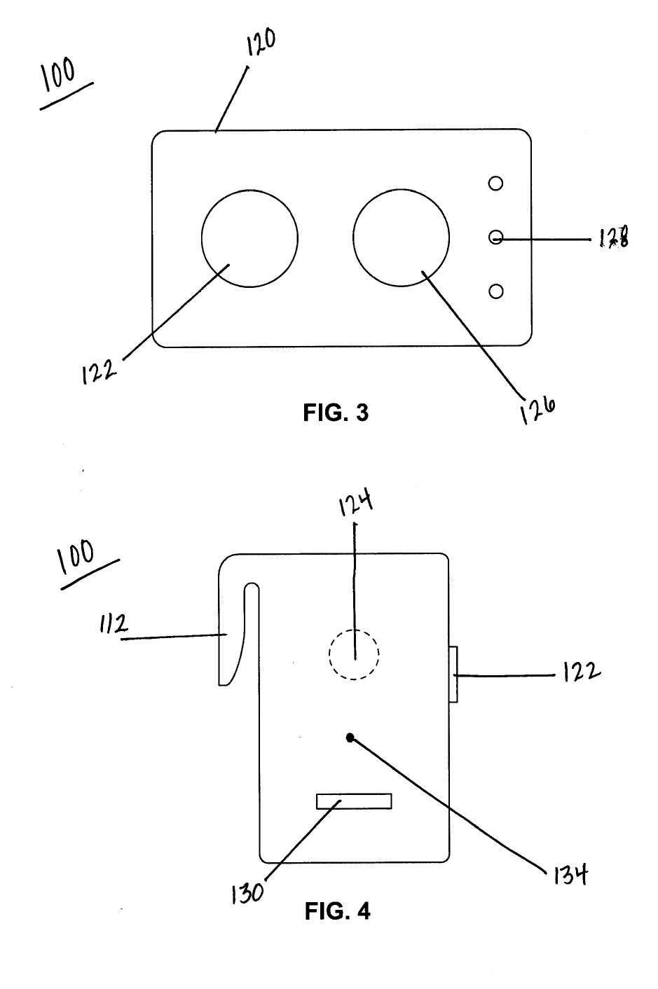

Referring now in more detail of FIGS. 3 and 4, a wearable device 100 for delivering external stimulation via a control panel 120 that attaches to a collar 102, belt, necklace, strap or other, via a clip 112 on the back. Control panel 120 components, mode of action 126 and benefits are similar to those detailed in FIG. 1. A benefit of this approach is the ease of use to attach, remove, recharge, and program control panel 120. Clip generator device 100 can attach to the collar, belt, necklace, strap or the like via many different ways, depending on the specific need. Control panel 120 includes speaker(s) 124, power button 122, mode selection button 126, indicator lights 128, and power button 122.

In one example use, the control panel 120 with clip 112 was attached to a collar and placed behind the neck and over the spine of an anxious, aged dog with inflammation in her back, (as shown by thermograph generated by an infrared imager). Embedded into control panel 120 was a mini vibration speaker that delivers both tactile and audio sensory vibrational stimulation through a range of frequencies. Frequencies that have proven effective range between 15 Hz and 300 Hz, and mimic very closely to those that are generated by tuning forks. Melodic music and recorded heartbeats played through the speakers also has calming effects. Therapy times range between 2 minutes and 20 minutes, with an average treatment time of about 10 minutes.

In the above canine example, 10 minutes of therapy that incorporated a frequency of 128 Hz with a recorded heartbeat at 60 beats per minute was administered. A visual post assessment of this dog showed she was much calmer, while a thermograph indicated her back had reduced inflammation, as seen by reduced temperature. By using the therapy on a regular, daily or frequent basis, the dog will not only stay calmer, but through nervous system activation and neuroplasticity, her pain levels will decrease and her mobile performance will improve.

Referring now in more detail of FIG. 5, a device 200 for delivering external energy stimulation via a control panel 220 embedded into a strap 202, belt, collar, or the like. A difference of this embodiment compared to FIG. 1 is the addition of speakers 224A and 224B external to control panel 220. A beneficial difference here is for animals such as horses or cows that have larger necks and a deeper spinal cord, hence device 200 can deliver energy more directly into the animal's ears.

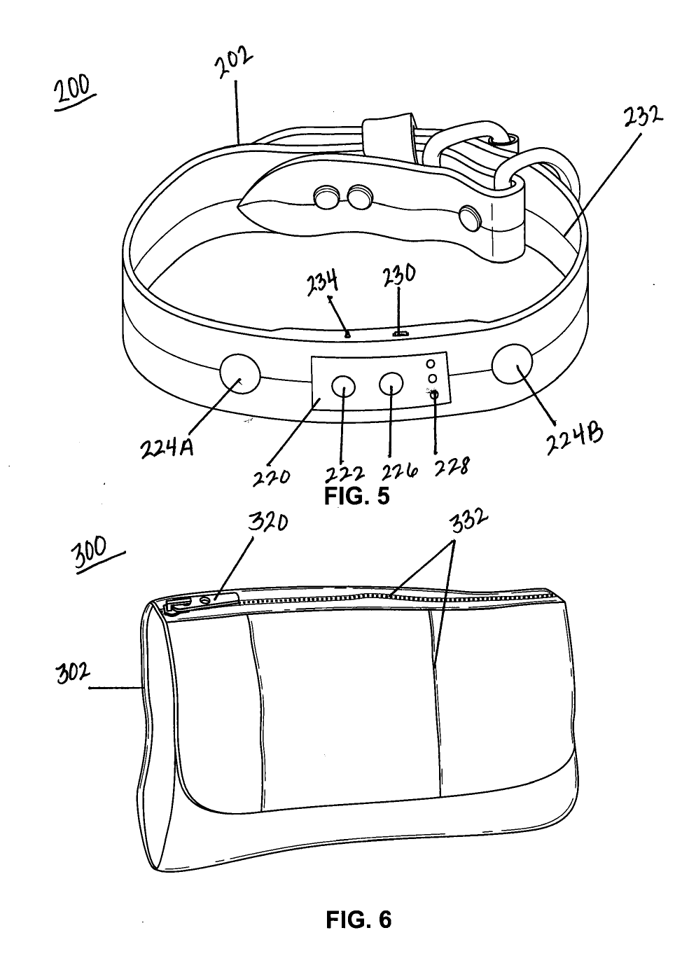

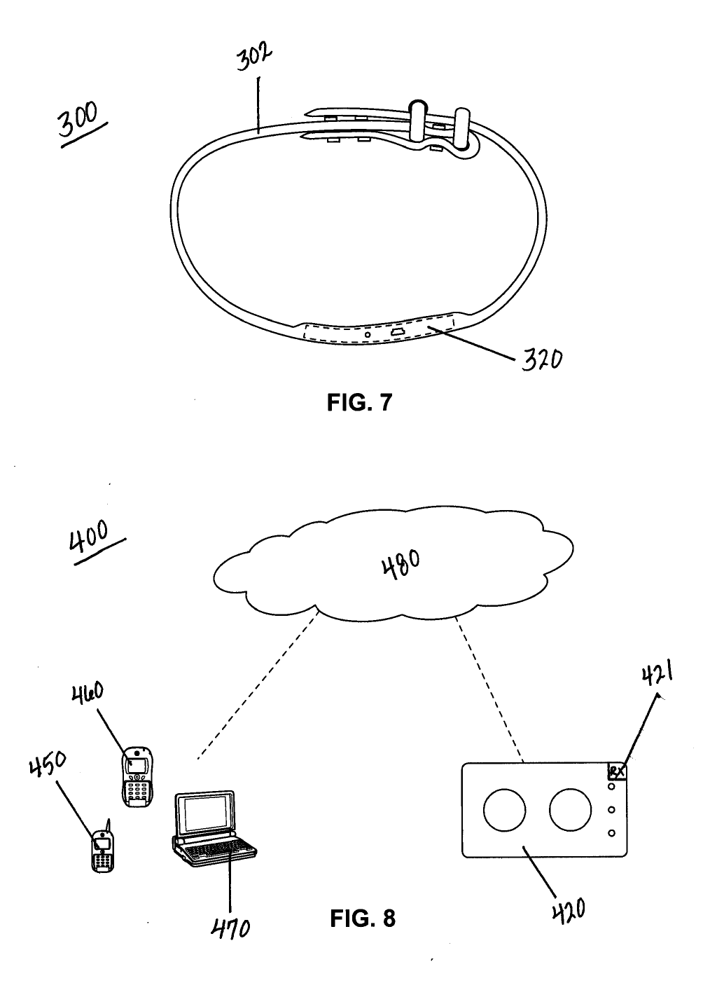

Referring now in more detail of FIGS. 6 and 7, is a device for delivering external energy stimulation 300 via a control panel 320 that may also include embedded conduction leads 332 into a shirt, harness, wrap 302, or similar device placed securely around the chest, leg, shoulder, neck or other part of a mammal. A beneficial difference for this device compared to FIG. 1 is the longer contact point of stimulation over the animal's spinal cord or directly to a specific area. In this example embodiment, device 300 is to be wrapped around the mammal's chest, leg, shoulder, or other affected part, and secured via a hook and loop system (also known as Velcro®) or other similar securing device.

Referring to FIG. 8, in more detail, is a system 400 for communicating with external animal devices, such as a control panel 420, via a remote 450, handheld 460, computer 470, wireless or other similar technology or device through the internet or cloud computing system 480. A wireless port or receiver 421 is embedded into control panel 420 and allows users to program, change, modify, start, stop or other function, the device or system. Wireless port 421, allows the user to control unit 420 from any location, via a handheld, remote, or other similar communication method and through internet 480.

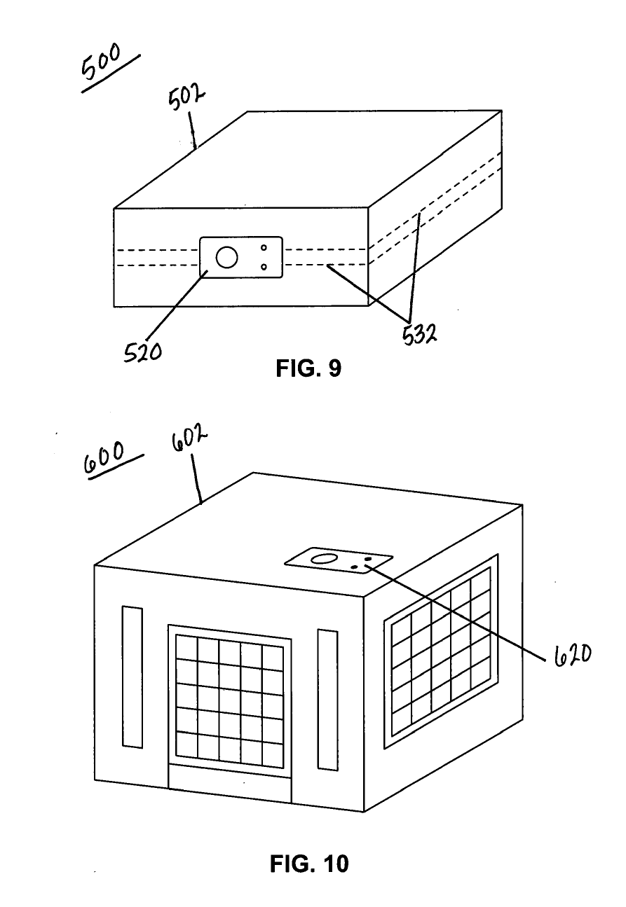

Referring to FIG. 9 in more detail, there is shown a system 500 for embedding or attaching a control unit 520 to a bed 502, blanket, mattress or similar surface where a mammal sleeps or lies down to rest. In this example embodiment, a therapy can be applied without physically attaching to a body. As in other delivery methods, internal leads 532 or other transmittal components can be placed inside the bedding to help deliver the treatment throughout.

Referring in greater detail to FIG. 10, there is shown a system 600 for incorporating or attaching a control panel 620 to a crate 602 or similar sleeping or resting enclosure. A benefit here is to deliver the therapy to the animal in a controlled setting without having to attach it to the animal, or making the animal sleep on a bedding with it included.

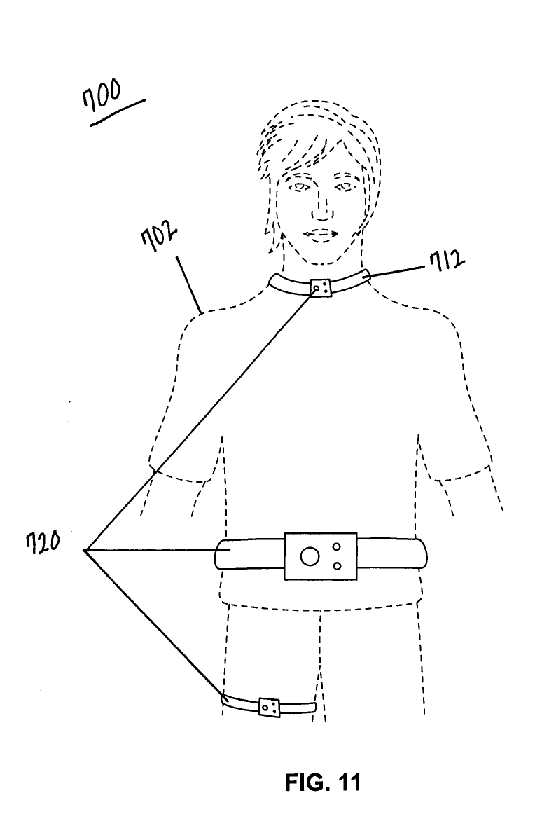

Referring in greater detail to FIG. 11, there is illustrated various ways 700 of attaching or embedding a control unit or clip 720 to humans 702 are illustrated. A clip can be attached to a collar 712, belt, waist or wrap that is on a human 702. A control unit 720 can also be embedded into a shirt collar, belt or wrap that is worn by a human. A benefit of these delivery methods is that a human can wear a therapeutic device and receive benefits when needed throughout the day or night. Although not shown in FIG. 11, collar or band 712 can also be worn around the head of human 702 to deliver one form of therapy to human 702.

In one example, the control clip was attached to a stretchable band and placed on a human behind the neck and over the spine. Embedded into the clip was a mini vibration speaker and audio control board to direct the therapy. The patient had recently strained his right anterior cruciate ligament (ACL) and had severe pain. Upon initial evaluation, he also demonstrated significant right-side weakness in his leg and arm, as well as high thermography images taken over his ACL.

After 5 minutes of applying the therapy of a frequency at 128 Hz and a relaxing melodic recording, a post review assessment was performed. A significant improvement in strength testing was observed, as well as decreased local thermograph temperature and reduction in pain. Continued daily or frequent use of the wearable therapy will help improve the ACL injury by helping and retraining the brain to send new healthy signals throughout the body.

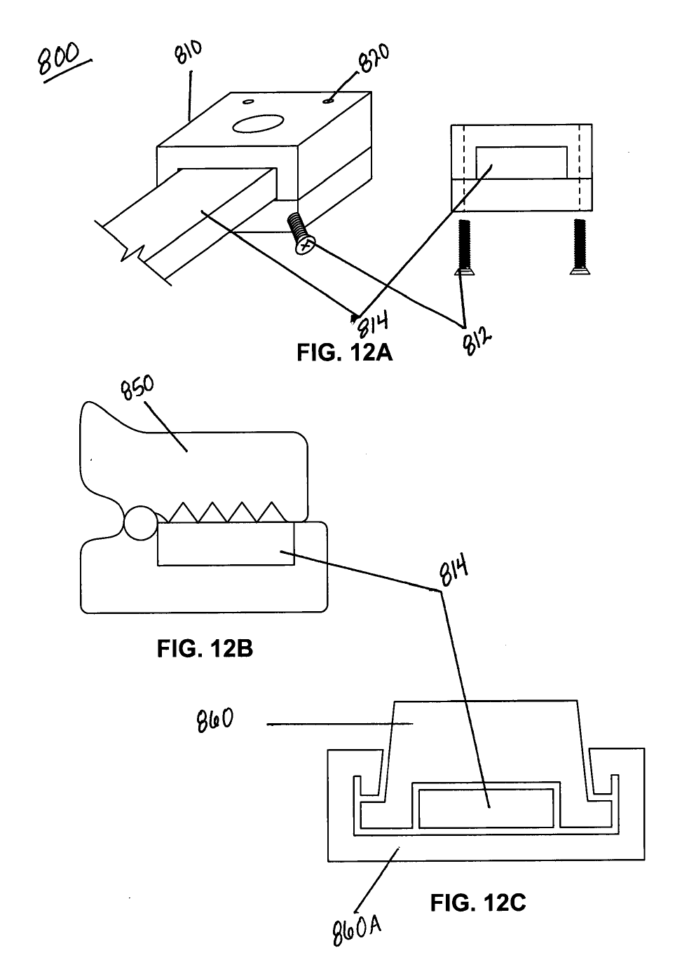

Referring to FIGS. 12A-12C in greater detail, various embodiments 800 are shown of clips 810, 850, and 860 that can be attached to belts, collars, bands 814, caps or similar areas. FIG. 12A shows a sandwich method 810 with a top and bottom piece, secured by screws 812 or the like. A benefit to this design is a control unit 820 is securely affixed to base unit 810 that uses screws 812 or other similar methods to keep the unit from detaching. FIG. 12B shows a clip 850 that hinges to open and securely close around band 814. A benefit of this design is that clip 850 can be easily removed and reattached to areas when and where needed. FIG. 12C shows a wrap around method of securely attaching a clip 860 without screws. A bottom piece 860A can be made of a pliable material. This design allows for secure attachment and easier removal versus the design with screws or snaps.

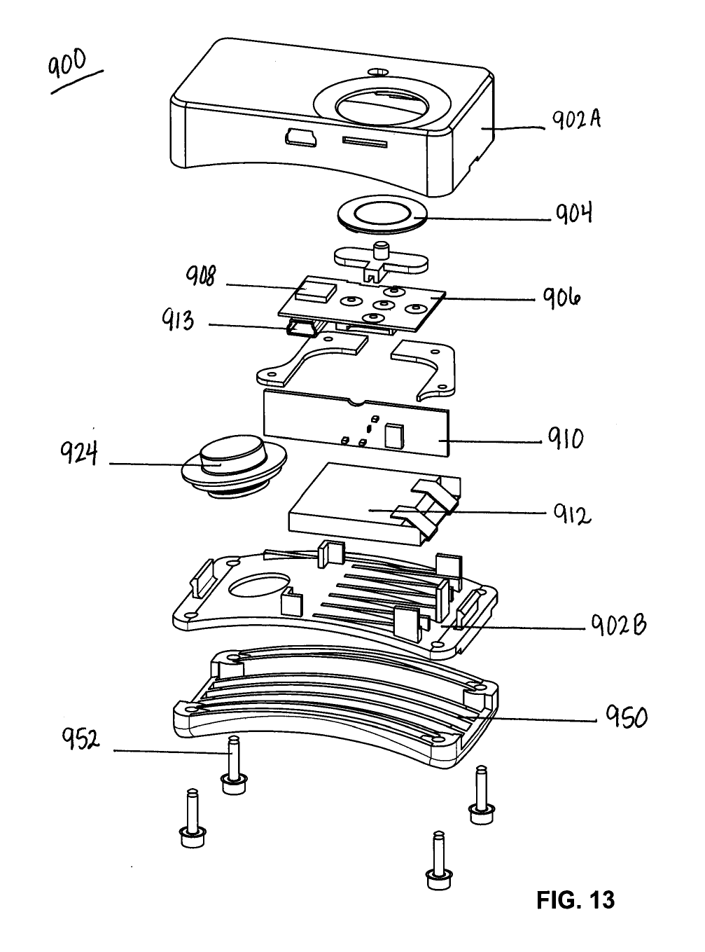

Referring now in greater detail to FIG. 13, there is a detailed view of the components that may be included in a control unit or clip 900. The unit 900 has an outer shell 902A and 902B made from plastic or metal, that may contain: a set of controls 904 and board 906 that may include volume or track controls; a memory card 908 that stores audio files; an amplification board to boost audio output 910; an internal battery or power source 912 and 913 that may have a way of externally interacting with it to recharge or may be removable to recharge; a wireless antenna to remotely interact with or control the device; regular or vibration speakers 924 or vibrating component with motor, or laser housing that delivers therapy; interaction pad or medium outlet that may be a different material to allow for better delivery of therapy to mammal; and a connection plate 950 and screws 952 to securely affix the unit or clip to a user.

By delivering a consistent and focused source of ongoing vibration, tones, audio, or laser to animals and mammals via our external stimulation methods, the following advantages and benefits can be seen: whole body vibration, external spinal stimulation, vibrational tones, audio frequencies, pulse, light and other forms of external energy which can help improve the lives of animals and humans by:
Increased flexibility and range of motion in muscles and joints and muscle strength by activating motor neurons as well improved coordination and stability.
Decreased time to healing for bones and other soft tissues.
Reduced anxiety, stress, depression, and other emotional triggers.
Reduced time to healing after surgery or trauma.
Prevention of long-term injury or illness and the reduction of chronic pain in joints and other areas.
Stimulating the body to recover quicker and increase muscle strength.
Increasing oxygenation to the body and the soft tissues.
Improving digestion.
Increase blood circulation and improved immune system
Improved athletic and physical performance.
Improved focus.
Cease barking, chewing, scratching, or other forms of outward aggression.
Increased body temperature—such as in cold weather or prior to performance, prior to or in lieu of a workout.
To burn calories and improve weight loss and tone.

The mammalian brain is receptor-activated and stimulated through various senses including touch, sound, light, smell and taste. The brain cortex having a large contribution of ipsilateral fibers that descend to synapse on the pontinemedulllary reticular formation and pontine areas explains one mode of operation for the invention in mammals. This area sends messages down to activate extensors and inhibit flexors. If this system fails, joints then loose support, flexors get too tight and nerve entrapment syndromes begin. This then leads to inflammation, which creates further weakness. When people or animals have chronic muscle pain, it is primarily in posterior muscle groups such as the extensors, triceps, trapezius, anterior legs and lower back. This then leads to more inflammation.

The brain again sends descending ipsilateral excitatory projections ultimately sending projections down to inhibit the sympathetic system, allowing for vasodilatation. Vibration follows peripheral sensory nerves to the dorsal horn of the spinal cord ascending with other sensory tracts to the medulla. From here the sensory information crosses over to the ventral posterior lateral nucleus of the thalamus and then is relayed to the samatosensory area of the brain in the parietal lobe that recognizes vibration. Information then crosses over to the motor strip of the cortex. By increasing activation or neuronal activity of the motor cortex, vibration energy transfers to electric energy increasing axonal output to the pontomedullary reticular formation in the brainstem. Activation of this area creates increased output to the extensor muscles creating increased strength to support muscles of legs, back, neck and shoulders. This will help with pain reduction and inhibit sympathetic output increasing vasodilation and parasympathetic activity. Added benefits include: increase oxygenation, better digestion, leading to faster recovery from injuries and surgeries.

Vibration therapy promotes the fracture healing in rabbits regardless of the frequency but in other mammals the frequency as well as the magnitude are important. Both bone strength and speed of fracture healing are better than those not receiving vibration therapy. In addition, bone strength is elevated by 20% approximately 30% by the best stimulation of stress. The best frequencies are 25 Hz and 50 Hz, while the second best are 12.5 and 100 Hz and then, 200 Hz. It has also been shown in rats that low magnitude, high frequency vibrational therapy promotes fracture healing (Clin Oral Implants Res. 2010 Oct 6. doi: 10.1111/j.I600-0501.2010.02020.x. [Epub ahead of print], which is herein incorporated by reference in its entirety).

Osteocytes are well evidenced to be the major mechanosensor in bone, responsible for sending signals to the effector cells (osteoblasts and osteoclasts) that carry out bone formation and resorption. Consistent with this hypothesis, it has been shown that osteocytes release various soluble factors (e.g. transforming growth factor-beta, nitric oxide, and prostaglandins) that influence osteoblastic and osteoclastic activities when subjected to a variety of mechanical stimuli, including fluid flow, hydrostatic pressure, and mechanical stretching. Recently, low-magnitude, high-frequency (LMHF) vibration (e.g., acceleration less than <1×g, where g=9.81 m/s(2), at 20-90 Hz) has gained much interest as studies have shown that such mechanical stimulation can positively influence skeletal homeostasis in animals and humans. (Bone. 2010 June; 46(6): 1508-15. Epub 2010 Mar 6. Effect of low-magnitude, high-frequency vibration on osteocytes in the regulation of osteoclasts. Lau E, Al-Dujaili S, Guenther A, Liu D, Wang L, You L. which is herein incorporated by reference in its entirety).

All of the following publications, literature and U.S. patent publications are incorporated herein by reference in their entirety: U.S. Pat. No. 8,123,707 Method and apparatus for connective tissue treatment; U.S. Pat. No. 8,121,696 Topical analgesia using electrical and vibration stimuli; U.S. Pat. No. 8,086,318 Portable assemblies, systems and methods for providing functional or therapeutic neurostimulation; U.S. Pat. No. 7,993,381 Method and apparatus for treating the body; U.S. Pat. No. 7,878,965 Therapeutic methods using electromagnetic radiation; U.S. Pat. No. 7,862,529 Neuromusculoskeletal knee support device; U.S. Pat. No. 7,860,573 Focused stimulation in a medical stimulation device; U.S. Pat. No. 7,783,348 Stimulation device for treating osteoarthritis; U.S. Pat. No. 7,762,953 Systems and methods for non-invasive physiological monitoring of non-human animals; U.S. Pat. No. 7,431,704 Apparatus and method for the treatment of tissue with ultrasound energy by direct contact; U.S. Pat. No. 7,416,536 Chest vibrating device; U.S. Pat. No. 7,340,874 Horse halter; U.S. Pat. No. 7,335,170 Therapeutic micro-vibration device; U.S. Pat. No. 7,252,051 Neck motion detector and method for bark control device; U.S. Pat. No. 7,211,055 System and method providing directional ultrasound therapy to skeletal joints; U.S. Pat. No. 7,207,953 Massage therapy vest; U.S. Pat. No. 7,108,663 Method and apparatus for cartilage growth stimulation; U.S. Pat. No. 6,885,305 System for locating and sending messages to pets; U.S. Pat. No. 6,851,385 Pet bed having orthopedic properties; U.S. Pat. No. 6,839,443 Bone conduction speaker; U.S. Pat. No. 6,746,473 Therapeutic laser device; U.S. Pat. No. 6,702,837 Therapeutic light device; U.S. Pat. No. 6,694,925 Thunderstorm fear-reducing cape for dogs; U.S. Pat. No. 6,620,117 Vibrational device for stimulating tissue and organs; U.S. Pat. No. 6,605,079 Method for performing lipoplasty using external laser radiation; U.S. Pat. No. 6,487,992 Dog behavior monitoring and training apparatus; U.S. Pat. No. 6,338,347 Blood circulation stimulator; U.S. Pat. No. 6,231,497 Magnetic therapy device; U.S. Pat. No. 6,073,589 Stimulation device and technique; U.S. Pat. No. 6,061,597 Method and device for healing bone fractures; U.S. Pat. No. 6,013,096 Hand-held laser light generator device; U.S. Pat. No. 5,815,077 Electronic collar for locating and training animals; U.S. Pat. No. 5,536,269 Bone and tissue lengthening device; U.S. Pat. No. 5,304,207 Electrostimulator with light emitting device; U.S. Pat. No. 5,255,327 Vibrostimulative device; U.S. Pat. No. 5,115,769 Vibrator; U.S. Pat. No. 5,101,810 Apparatus and method for therapeutic application of vibro-acoustical energy to human body; U.S. Pat. No. 5,035,235 Music vibration table and system; U.S. Pat. No. 4,920,966 Headphone type illuminating device with massage; U.S. Pat. No. 4,144,893 Neuromuscular therapy device; U.S. Pat. No. 2,715,901 Vibrating contour chair; U.S. Pat. No. 2,694,394 Mechanical vibrator for therapeutic use; U.S. Design Pat. No. D520,894 Collar-mounted bark limiting device; U.S. Patent Publication No. 2010/0174217 Point vibration therapy device; U.S. Patent Publication No. 2008/0077192 System and method for neuro-stimulation; and U.S. Patent Publication No. 2008/0249439 Treatment of inflammation by non-invasive stimulation.

### ADDITIONAL REFERENCES

### Clinical Articles

Vermiere, Simon et al. Regional Cerebral Blood Flow Changes in Dogs with Anxiety, Measured with SPECT. Brain Imaging and Behavior. 3:342-349. 2009.
Schortinghuis, J. et al. Ultrasound Stimulation of Maxillofacial Bone Healing. Critical Reviews in Oral Biology and Medicine. 14(1):63-74. 2003.
Prisby, Rhonda et al. Effects of Whole Body Vibration on the Skeleton and other Organ Systems in Man and Animal Models: What we Know and What we Need to Know. Ageing Research Reviews. 7:319-329. 2008.
Chen, L P et al. The Effects of Frequency of Mechanical Vibration on Experimental Fracture Healing.
Shi, H F at al. Low-magnitude High-frequency Vibration Treatment Augments Fracture Healing in Ovariectomy-induced Osteoporotic Bone. Bone. 46(5):1299-305. 2010.
Leung, K S et al. Low-magnitude High-frequency Vibration Accelerates Callus Formation, Mineralization, and Fracture Healing in Rats. Journal of Orthopedic Research. 27(4):458-65. 2009.
Ogawa, T et al. The Effect of Whole-Body Vibration on Peri-Implant Bone Healing in Rats. Clin Oral Implants Research. 2010.
Lohman, E B et al. The Effect of Whole Body Vibration on Lower Extremity Skin Blood Flow in Normal Subjects. Medical Science Monitor. 13(2):CR71-6. 2007.
Stengel, S V et al. Effects of Whole Body Vibration Training on Different Devices on Bone Mineral Density. Medicine & Science in Sports & Exercise. 2010.
Rauch, F et al. Reporting Whole-Body Vibration Intervention Studies: Recommendations of the International Society of Musculoskeletal and Neuronal Interactions. 10(3): 193-8. 2010.
Rauch, F. Vibration Therapy. Developmental Medicine and Child Neurology. Suppl 4:166-8. 2009.
Kerschan-Schindl, K et al. Whole-body Vibration Exercise Leads to Alterations in Muscle Blood Volume. Clinical Physiology. 21(3):377-82. 2001.
Boyd-Brewer, C. Vibroacoustic Sound Therapy Improves Pain Management and More. Holistic Nursing Practice. May/June. 111-118. 2004.Ultrasound Stimulation of Maxillofacial Bone Healing, Critical Reviews in Oral Biology & Medicine, 14(I):63-74 (2003).

### Books

Albasini, A et al. Using Whole Body Vibration in Physical Therapy and Sport: Clinical Practice and Treatment Exercises. 2010.
Peirce, P et al. Frequency: the Power of Personal Vibration. 2009.
Krames, E et al. Neuromodulation: Volume 1. 2009.
Hughes, J. Pain Management: From Basics to Clinical Practice. 2008.
Bourne, E. Natural Relief for Anxiety. 2009.
Bruscia, K. Defining Music Therapy. 1998.
Becker, R. The Body Electric. 1998.
The Soundbeam Project. What is Vibroacoustic Therapy?

This written description of the invention enables one of ordinary skill to make and use what is presently described. A person of ordinary skill should understand and appreciate that there are variations and combinations to the methods described herein, and should therefore not limit the invention to what is described, but by all the embodiments and methods within the scope and spirit of the invention.

We claim:

1. An apparatus for delivering therapeutic energy to at least a portion of a mammal comprising:
an energy module adapted to generate energy waves in an energy range configured to provide a therapeutic effect on a mammal; and
means for supporting the energy module at a treatment site, the energy module being coupled to said supporting means.

2. The apparatus of claim 1 wherein said supporting means is selected from a group consisting of a collar, strap, headband, wrap, shirt, halter, vest, crate, cage, blanket, mattress, board and wearable clip.

3. The apparatus of claim 1 wherein an energy type for the energy waves is selected from a group consisting of radio waves, low power light waves, low power infrared waves, ultrasonic waves, audio and mechanical waves.

4. The apparatus of claim 1 wherein the energy type is a high frequency, low magnitude energy adapted to bring about vibration in the mammal's tissue or bone.

5. A method for delivering therapeutic energy to at least a portion of a body comprising the steps of:
providing an energy output module adapted to generate energy waves in an energy range configured to provide a therapeutic effect on a mammal; and
directing the energy waves from the energy output module at a treatment site of the mammal for a predetermined period of time.

6. The method of claim 5 further including the step of coupling the energy output module to a delivery member adapted to enhance a therapeutic effect of the energy waves to the mammal.

7. The method of claim 5 further including the step of determining an energy level within the energy range as a function of a desired treatment.

8. The method of claim 5 further including the step of selecting an energy type for the energy waves from a group consisting of radio waves, low power light waves, low power infrared waves, ultrasonic waves, audio waves.

9. The method of claim 6 further including the step of selecting the delivery member from a group consisting of a collar, strap, headband, wrap, shirt, halter, vest, crate, cage, blanket, mattress, board and wearable clip.

10. A system for remotely providing a therapeutic treatment to a mammal comprising.

11. an energy module adapted to generate energy waves in an energy range configured to provide a therapeutic effect on a mammal;
means for supporting the energy module at a treatment site, the energy module being coupled to said supporting means; and
means for remotely and wirelessly communicating with the energy module, wherein said energy module is configured to be responsive to said communication means such that a treatment is delivered to the mammal for a predetermined time.
